(19) United States
(12) Patent Application Publication
(10) Pub. No.: US 2005/0097682 A9
(43) Pub. Date: May 12, 2005

(54) Oxidation dyeing composition based on 1-(4-aminophenyl)pyrrolidines substituted in positions 2 and 4

(76) Inventors: Eric Terranova, Magagnose (FR); Stephane Sabelle, Paris (FR); Laurent Vidal, Paris (FR)

Correspondence Address: Finnegan Henderson Farabow;Garrett & Dunner, 1300 I Street NW, Washington, DC, 20005, US

(21) Appl. No.: 10/433,411

(22) PCT Filed: Nov. 14, 2001
(86) PCT No.: PCT/FR01/03571

(30) Foreign Application Priority Data
Dec. 6, 2000 (FR) 0015843

(51) Int. Cl. A61K 7/13

(52) U.S. Cl. 8/405

(57) ABSTRACT

The subject of the invention is a composition for the oxidation dyeing of keratinous fibers, and in particular human keratinous fibers such as hair, comprising, as oxidation base, a 1-(4-aminophenyl)pyrrolidine substituted at the 2- and 4-positions. The subject of the invention is also the method for the oxidation dyeing of keratinous fibers using these compositions.

The subject of the invention is a composition for the oxidation dyeing of keratinous fibers, and in particular human keratinous fibers such as hair, comprising, as oxidation base, a 1-(4-aminophenyl)pyrrolidine substituted at least at the 2- and 4-positions.

It is known to dye keratinous fibers, and in particular human hair, with dyeing compositions containing oxidation dye precursors, in particular para-phenylenediamines, ortho- or para-aminophenols, heterocyclic compounds such as diaminopyrazole derivatives, pyrazolo[1,5-a]pyrimidine derivatives, pyrimidine derivatives, pyridine derivatives, 5,6-dihydroxyindole derivatives, 5,6-dihydroxyindoline derivatives generally called oxidation bases. Oxidation dye precursors, or oxidation bases, are colorless or weakly colored compounds which, combined with oxidizing products, can give rise, by a process of oxidative condensation, to colored and coloring compounds.

It is also known that the shades obtained with these oxidation bases can be varied by combining them with couplers or color modifiers, the latter being chosen in particular from aromatic meta-diamines, meta-aminophenols, meta-hydroxyphenols and certain heterocyclic compounds such as, for example, pyrazolo[1,5-b]-1,2,4-triazole derivatives, pyrazolo[3,2-c]-1,2,4-triazole derivatives, pyrazolo[1,5-a]pyrimidine derivatives, pyridine derivatives, pyrazol-5-one derivatives, indoline derivatives and indole derivatives.

The variety of the molecules used in oxidation bases and couplers makes it possible to obtain a rich palette of colors.

The so-called “permanent” color obtained using these oxidation dyes must moreover meet a number of requirements. Thus, it must be without drawbacks from the toxicological point of view, it must make it possible to obtain shades in the desired intensity and exhibit good resistance to external agents (light, adverse weather conditions, washing, permanent waving, perspiration, rubbing).

The dyes must also make it possible to cover gray hair, and be the least selective possible, that is to say make it possible to obtain the smallest possible differences in color right along the same keratinous fiber, which may indeed be differently sensitized (i.e. damaged) between its tip and its root. They must also exhibit good chemical stability in the formulations. They must also exhibit a good toxicological profile.

In the field of hair dyeing, para-phenylenediamine and para-toluenediamine are widely used oxidation bases. They make it possible to obtain a variety of shades with oxidation couplers.

However, a need exists to discover novel oxidation bases exhibiting a better toxicological profile than para-phenylenediamine and para-toluenediamine, while making it possible to confer on the hair excellent properties of color intensity, shade variety, color uniformity and fastness to external agents.

It has already been proposed, in particular in patent application GB 2 239 265, to use 2-(β-hydroxyethyl)-para-phenylenediamine or N,N-bis(β-hydroxyethyl)-para-phenylenediamine as potential replacements for para-phenylenediamine and para-tolylenediamine. It is likewise the case for 2-(hydroxyalkoxy)-para-phenylenediamines (see in particular U.S. Pat. No. 5,538,516).

However, N,N-bis(β-hydroxyethyl)-para-phenylenediamine and 2-(β-hydroxyethyl)-para-phenylenediamine have the disadvantage of giving a more limited variety of shades and of giving less color intensity and less uniformity to the hair than para-phenylenediamine and 4-amino-2-methylaniline. It is likewise the case for 2-(hydroxyalkoxy)-para-phenylenediamines which give the hair color which evolves and changes over time.

Moreover, it is known from the literature (R. L. Bent et al., J.A.C.S. 73, 3100, 1951) that 4-para-phenylenediamine derivatives in which one of the nitrogen atoms is contained in a nonaromatic 6- or 7-membered carbon or heterocyclic ring are less oxidizable than 4-para-phenylenediamine derivatives in which one of the nitrogen atoms is substituted with two disymmetric substituents, which are themselves less oxidizable than para-phenylenediamine derivatives in which one of the nitrogen atoms is substituted with two symmetric substituents.

It is also mentioned in the same article that the para-phenylenediamine derivative in which one of the nitrogen atoms is contained in a nonaromatic 5-membered carbon ring is more oxidizable than each of the derivatives cited above. This particular class of N-(4-aminophenyl)-pyrrolidine derivatives therefore makes it possible to obtain condensation reactions with couplers in an oxidizing medium whose kinetics is accelerated compared with the para-phenylenediamine derivatives cited above.

However, oxidation bases which are too oxidizable and which react with couplers according to accelerated reaction rates generally lead to the formation of dyes outside the keratinous fiber. The intensities, the fastness and the uniformity of the colorations thus obtained on the hair are generally insufficient.

However, U.S. Pat. No. 5,851,237 proposes the use of 1-(4-aminophenyl)pyrrolidine derivatives optionally substituted on the benzene nucleus in order to replace para-phenylenediamine. In this regard, the same patent proposes very preferentially the use of 1-(4-aminophenyl)pyrrolidone as the substitute for para-phenylenediamine.

Now, it is known from the literature that 1-(4-aminophenyl)pyrrolidine possesses a high allergenic activity (R. L. Bent et al., J.A.C.S. 73, 3100, 1951).

U.S. Pat No. 5,993,491 proposes the use of N-(4-aminophenyl)-2-hydroxymethylpyrrolidine derivatives optionally substituted on the benzene nucleus and on the pyrrolidine heterocycle at the 4-position with a hydroxyl radical in order to replace para-phenylenediamine. As preferred compounds, said patent proposes N-(4-aminophenyl)-2-(hydroxymethyl)pyrrolidine substituted with a hydrogen atom or a methyl radical at the 3-position. However, it has been clearly established that these compounds do not make it possible to give the hair a coloration of equivalent quality to that obtained with para-phenylenediamine or with para-toluenediamine because of the lack of intensity and of uniformity of the color.

Patent application JP 11158048 proposes hair dyeing compositions which offer good properties of spreading, ease of application and resistance to shampoo. These compositions contain at least one compound chosen from 4-aminoaniline derivatives optionally substituted on the benzene nucleus and in which one of the nitrogen atoms is contained in a 5- to 7-membered carbon ring or at least one compound chosen from 4-aminoaniline derivatives optionally substituted on the benzene nucleus and in which one of the nitrogen atoms is substituted with a radical Z₁ and a radical Z₂, Z₁ being an alkyl, aryl or heterocyclyl group, and Z₂ being a radical —(CH₂—CH₂—O)-Z₃ where Z₃ represents a hydrogen atom, an alkyl, aryl or heterocyclyl group.

In terms of dyeing power, ease of application, uniformity of color and resistance to shampoo, this patent application demonstrates that the preferred derivatives N-(3-isopropoxy-4-aminophenyl)-2,5-dimethylpyrrolidine, 1-(3-methyl-4-aminophenyl)-2,5-dihydroxyethylpyrrolidine, N-(3-methyl-4-aminophenyl)-3-(2-hydroxyethyloxy)pyrrolidine and N-(3-methyl-4-aminophenyl)-2-methyl-4-hydroxypyrrolidine behave like oxidation bases equivalent to para-phenylenediamine derivatives in which the nitrogen atom is contained in a functionalized 6-membered piperidine ring.

Now, it is known that when one of the. nitrogen atoms of the para-phenylenediamine derivatives is contained in a 6-membered ring, in particular a piperidine ring, the activation energy to lead to the corresponding quinoneimine oxidized form is among the highest of the N,N-disubstituted para-phenylene-diamines. Consequently, the oxidative condensation reactions of such bases with or without couplers are kinetically and energetically unfavorable and the dyeing compositions containing such oxidation bases give the hair insufficient properties in terms of intensity and uniformity of color compared with those containing para-phenylenediamine or para-toluene-diamine.

The result is that the compositions containing para-phenylenediamine derivatives having a nitrogen atom contained in a functionalized pyrrolidine ring as described in patent application JP 11158048 do not make it possible to give the hair dyeing results equivalent to those obtained with para-phenylenediamine or para-toluenediamine.

It is therefore clear that there is a real need to discover novel oxidation bases having both a good toxicological profile and properties such that the compositions containing them make it possible to confer on the hair excellent properties of intensity of color, variety of shades, uniformity of color and fastness toward various external attacks to which the hair may be subjected.

The aim of the present invention is to develop novel dyeing compositions which do not have the disadvantages of the oxidation bases of the prior art.

This aim is achieved with the present invention whose subject is a composition for the oxidation dyeing of keratinous fibers, and in particular human keratinous fibers such as hair, comprising, in a medium appropriate for dyeing,
at least one oxidation base chosen from compounds of the following formula (I), and or their addition salts with an acid 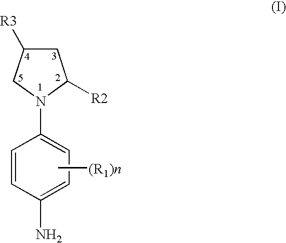
   in which:
R represents a halogen atom; a linear or branched C₁-C₇ carbon chain, which is saturated or which may contain one or more double bonds and/or one or more triple bonds, which may be in the form of a 3- to 6-membered ring, it being possible for one or more carbon atoms of the chain to be replaced by an oxygen, nitrogen or sulfur atom, by an SO₂ group or by a halogen atom, the radical R₁ not containing a peroxide bond, or a diazo, nitro or nitroso radical;
R₂ represents a saturated or unsaturated C₁-C₄ carbon chain; a C₁-C₄ alkyl radical substituted with a C₁-C₄ alkoxy radical, an acetoxy radical, an amino radical, a carboxyl radical, a carbamoyl radical, a (C₁-C₄) mono- or dialkylcarbamoyl radical, a (C₁-C₄) alkoxycarbonyl radical, a C₁-C₆ monohydroxyalkoxy radical or with a C₂-C₆ polyhydroxyalkoxy group; a C₁-C₄ monohydroxyalkyl radical; a C₁-C₆ polyhydroxyalkyl radical; a C₁-C₄ aminoalkyl radical in which the amine is mono- or disubstituted with a C₁-C₄ alkyl radical, an acetyl radical, a C₁-C₄ monohydroxyalkyl radical or a C₂-C₆ polyhydroxyalkyl radical; a C₂-C₄ polyaminoalkyl radical; a C₂-C₄ alkyl radical substituted with at least one amino group and at least one hydroxyl group; a carboxyl radical; a carbamoyl radical; a (C₁-C₄) mono- or dialkylcarbamoyl radical; a (C₁-C₄)alkoxycarbonyl radical; a (C₁-C₄)alkylcarbonyl radical,
R₃ represents a hydroxyl radical; an amino radical; a saturated or unsaturated C₁-C₄ carbon chain; a radical —OR₄ in which R₄ represents a saturated or unsaturated C₁-C₄ alkyl radical which may be substituted with one or more radicals chosen from the group consisting of a halogen atom, a hydroxyl, a C₁-C₂ alkoxy, an amino or a C₁-C₂ aminoalkyl radical; a radical —NR₅R₆ in which R₅ and R₆ represent, independently of each other, a hydrogen atom, a C₁-C₄ alkyl radical, a C₁-C₄ alkyl radical substituted with one or more radicals chosen from the group consisting of a halogen atom, a hydroxyl, a C₁-C₂ alkoxy, an amino or a C₁-C₂ aminoalkyl radical.
n is between 0 and 2, it being understood that when n is equal to 2, then the radicals R, may be identical or different,
   with the exception of the compound 1-(3-methyl-4aminophenyl)-4-hydroxy-2-methylpyrrolidone and the compound 1-(4-aminophenyl)-4-hydroxy-2-hydroxymethylpyrrolidone.

Completely unexpectedly and surprisingly, the 1-(4-aminophenyl)pyrrolidine derivatives of formula (I) substituted at least at the 2- and 4-position of the pyrrolidine ring may be used as oxidation dye precursors, and in addition make it possible to obtain dyeing compositions which give intense colorations of keratinous fibers and which exhibit good resistance with respect to external agents (light, adverse weather conditions, washing, permanent waving, perspiration, rubbing). Finally, these compounds are found to be easily synthesizable and are chemically stable.

Unless otherwise stated, the radicals, groups, or carbon chains defined above in formula (I) may be linear or branched.

According to the invention, when it is indicated that one or more of the carbon atoms of the radical R₁ may be replaced by an oxygen, nitrogen or sulfur atom or by an SO₂ group, and/or that said radical R₁ may contain one or more double bonds and/or one or more triple bonds, that means that it is possible, by way of example, to carry out the following conversions:

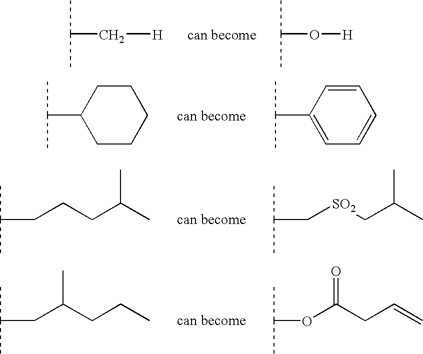

In formula (I), the radical R₁ is preferably chosen from a chlorine or bromine atom, a methyl, ethyl, isopropyl, vinyl, allyl, methoxymethyl, hydroxymethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy, or 2-hydroxyethyloxy radical. According to a particular embodiment, R₁ is chosen from a methyl, hydroxymethyl, 2-hydroxyethyl, 1,2-dihydroxyethyl, methoxy, or 2-hydroxyethoxy radical, and preferably a methyl, hydroxymethyl or 1,2-dihydroxyethyl radical.

According to a particular embodiment, n is equal to 0 or 1. When n is equal to 1, then R is preferably at the 3-position of the benzene ring.

The radical R₂ of formula (I) is preferably chosen from the hydroxymethyl radical, aminomethyl radical, carboxyl radical, carbamoyl radical, 2-hydroxyethyloxymethyl radical, 2-hydroxyethylaminomethyl radical, and methoxymethyl.

The radical R₃ of formula (I) is preferably chosen from a hydroxyl radical, an acetoxy radical, an amino radical, a methylamino radical, a dimethylamino radical, a hydroxyethylamino radical, a hydroxyethyloxy radical, and an acetamido radical.

According to a particular embodiment, R₃ is a hydroxyl, amino, 2-hydroxyethylamino or 2-hydroxyethyloxy radical and R₂ is chosen from a carboxyl, carbamoyl, aminomethyl, 2-hydroxyethyloxymethyl, 2-hydroxyethylaminomethyl or methoxymethyl radical.

In formula (I), the asymmetric carbons substituted with the radicals R₂ and R₃ may be, independently of each other, of the (R) and/or (S) configuration.

Among the compounds of formula (I) which are used for the present invention, there may be mentioned in particular:

| Formula | Nomenclature | Formula | Nomenclature |
|---|---|---|---|
| 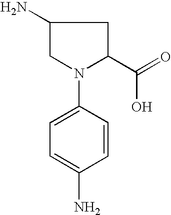 | 4-Amino-1-(4-amino-phenyl)- pyrrolidine-2-carboxylic acid | 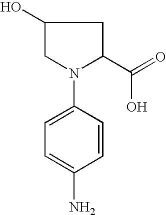 | 1-(4-Amino-phenyl)-4-hydroxy- pyrrolidine-2-carboxylic acid |
| 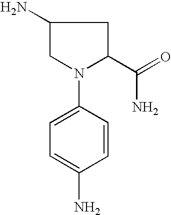 | 4-Amino-1-(4-amino-phenyl)- pyrrolidine-2-carboxylic acid amide | 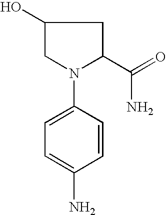 | 1-(4-Amino-phenyl)-4-hydroxy- pyrrolidine-2-carboxylic acid amide |
| 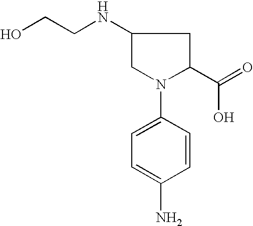 | 1-(4-Amino-phenyl)-4-(2- hydroxy-ethylamino)- pyrrolidine-2-carboxylic acid | 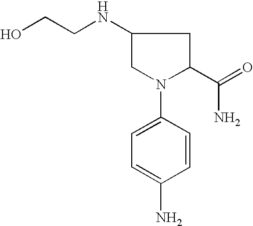 | 1-(4-Amino-phenyl)-4-(2- hydroxy-pyrrolidine-2- carboxylic acid amide |
| 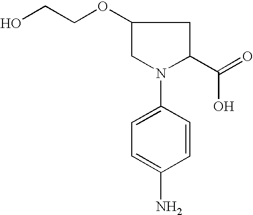 | 1-(4-Amino-phenyl)-4-(2- hydroxy-ethoxy)-pyrrolidine- 2-hydroxy-ethoxy)- pyrrolidine-2-carboxylic acid | 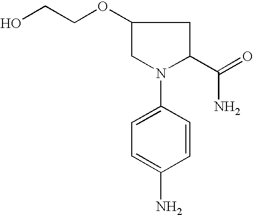 | 1-(4-Amino-phenyl)-4-(2- hydroxy-ethoxy)-pyrrolidine-2- carboxylic acid amide |
| 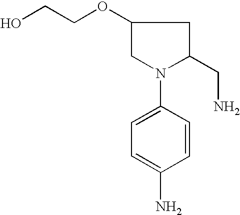 | 2-[5-Aminomethyl-1-(4- amino-phenyl)-pyrrolidin-3- yloxy]-ethanol | 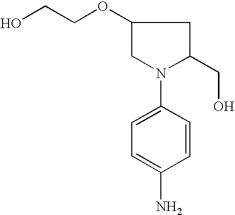 | 2-[1-(4-Amino-phenyl)-5- hydroxymethyl-pyrrolidin-3- yloxy]-ethanol |
| 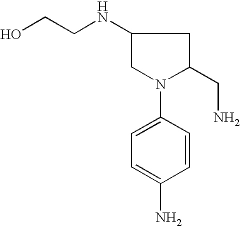 | 2-[5-Aminomethyl-1-(4- amino-phenyl)-pyrrolidin-3- ylamino]-ethanol | 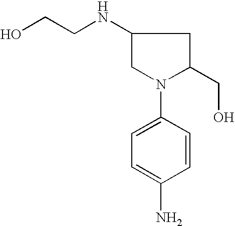 | 2-[1-(4-Amino-phenyl)-5- hydroxymethyl-pyrrolidin-3- ylamino]-ethanol |
| | | 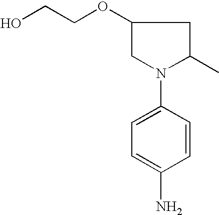 | 2-[1-(4-Amino-phenyl)-5- methyl-pyrrolidin-3-yloxy]- ethanol |
| 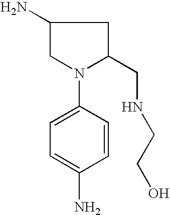 | 2-{[4-Amino-1-(4-amino- phenyl)-pyrrolidin-2-ylmethyl]- amino}-ethanol | 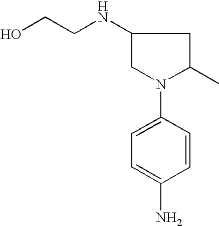 | 2-[1-(4-Amino-phenyl)-5- methyl-pyrrolidin-3-ylamino]- ethanol |
| 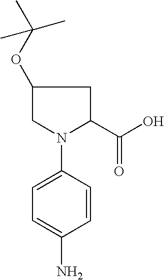 | 1-(4-Amino-phenyl)-4-tert- butoxy-pyrrolidine-2- carboxylic acid | 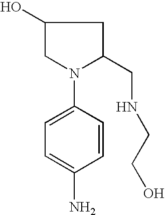 | 1-(4-Amino-phenyl)-5-[(2- hydroxy-ethylamino)-methyl]- pyrrolidin-3-ol |
| 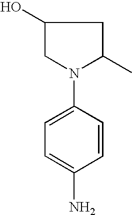 | 1-(4-Amino-phenyl)-5- methyl-pyrrolidin-3-ol | 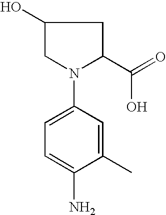 | 1-(4-Amino-3-methyl-phenyl)- 4-hydroxy-pyrrolidine-2- carboxylic acid |
| 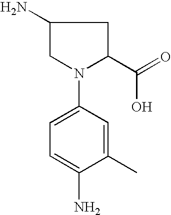 | -Amino-1-(4-amino-3-methyl- phenyl)-pyrrolidine-2- carboxylic acid | 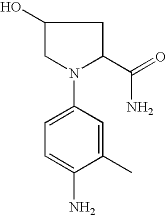 | 1-(4-Amino-3-methyl-phenyl)- 4-hydroxy-pyrrolidine-2- carboxylic acid amide |
| 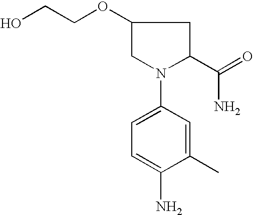 | 1-(4-Amino-3-methyl- phenyl)-4-(2-hydroxy- ethoxy)-pyrrolidine-2- carboxylic acid amide | 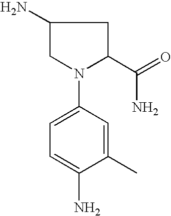 | 4-Amino-1-(4-amino-3- methyl-phenyl)-pyrrolidine-2- carboxylic acid amide |
| 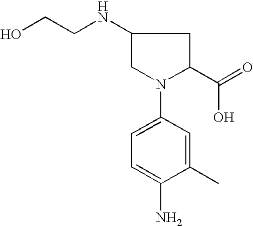 | 1-(4-Amino-3-methyl- phenyl)-4-(2-hydroxy- ethylamino)-pyrrolidine-2- carboxylic acid | 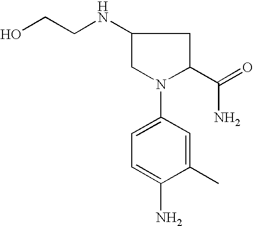 | 1-(4-Amino-3-methyl- phenyl)-4-(2-hydroxy- ethylamino)-pyrrolidine-2- carboxylic acid amide |
| 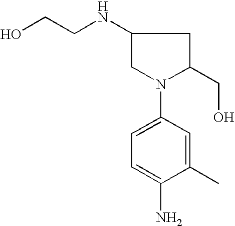 | 2-[1-(4-Amino-3-methyl- phenyl)-5-hydroxymethyl- pyrrolidin-3-ylamino]-ethanol | 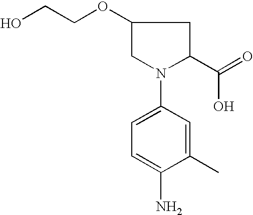 | 1-(4-Amino-3-methyl-phenyl)- 4-(2-hydroxy-ethoxy)- pyrrolidine-2-carboxylic acid |
| 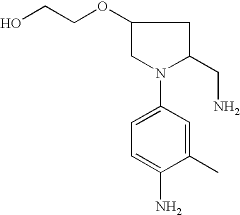 | 2-[5-Aminomethyl-1-(4- amino-3-methyl-phenyl)- pyrrolidin-3-yloxy]-ethanol | 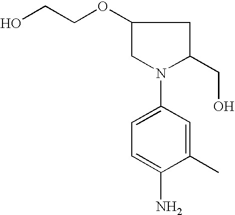 | 2-[1-(4-Amino-3-methyl- phenyl)-5-hydroxymethyl- pyrrolidin-3-yloxy]-ethanol |
| 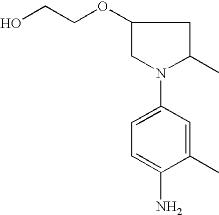 | 2-[1-(4-Amino-3-methyl- phenyl)-5-methyl-pyrrolidin- 3-yloxy]-ethanol | 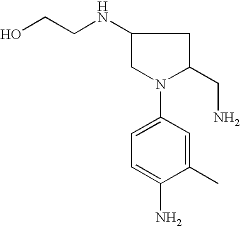 | 2-[5-Aminomethyl-1-(4-amino- 3-methyl-phenyl)-pyrrolidin-3- ylamino]-ethanol |
| 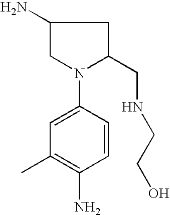 | 2-{[4-Amino-1-(4-amino-3- methyl-phenyl)-pyrrolidin-2- ylmethyl]-amino}-ethanol | 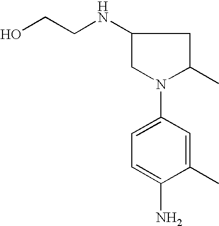 | 2-[1-(4-Amino-3-methyl- phenyl)-5-methyl-pyrrolidin-3- ylamino]-ethanol |
| 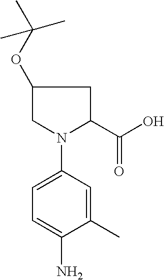 | 1-(4-Amino-3-methyl- phenyl)-4-tert-butoxy- pyrrolidine-2-carboxylic acid | 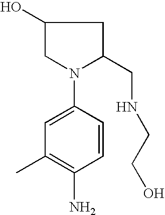 | 1-(4-Amino-3-methyl-phenyl)-5- [(2-hydroxy-ethylamino)- methyl]-pyrrolidin-3-ol |

and their addition salts with an acid.

The addition salts with an acid of the compounds of formula (I) in accordance with the invention are preferably chosen from the inorganic or organic salts such as the hydrochlorides, hydrobromides, sulfates, citrates, succinates, tartrates, lactates and acetates. The hydrochlorides are particularly preferred.

The para-phenylenediamine derivatives with a pyrrolidinyl group of formula (I), and their methods of synthesis are known; see in particular patent application DE 4 241 532 (AGFA).

The compound(s) of formula (I) in accordance with the invention preferably represent from 0.0005 to 12% by weight approximately of the total weight of the dyeing composition, and still more preferably from 0.005 to 6% by weight approximately of this weight.

The medium appropriate for dyeing (or carrier) generally consists of water or of a mixture of water and of at least one organic solvent to solubilize the compounds which might not be sufficiently soluble in water. As organic solvent, there may be mentioned for example lower C₁-C₄ alkanols, such as ethanol and isopropanol; polyols or polyol ethers such as 2-butoxyethanol, propylene glycol, monomethyl ether of propylene glycol, monoethyl ether and monomethyl ether of diethylene glycol, as well as aromatic alcohols such as benzyl alcohol or phenoxyethanol, similar products and mixtures thereof.

The solvents may be present in proportions preferably of between 1 and 40% by weight approximately relative to the total weight of the dyeing composition, and still more preferably between 5 and 30% by weight approximately.

The pH of the dyeing composition in accordance with the invention is generally between 3 and 12 approximately, and preferably between 5 and 11 approximately. It can be adjusted to the desired value by means of acidifying or alkalinizing agents normally used in dyeing keratinous fibers.

Among the acidifying agents, there may be mentioned, by way of example, inorganic or organic acids such as hydrochloric acid, orthophosphoric acid, sulfuric acid, carboxylic acids such as acetic acid, tartaric acid, citric acid, lactic acid and sulfonic acids.

Among the alkalinizing agents, there may be mentioned, by way of example, aqueous ammonia, alkali metal carbonates, alkanolamines such as mono-, di- and triethanolamines as well as derivatives thereof, sodium or potassium hydroxides and the compounds of the following formula (II): 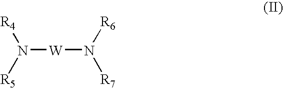
in which W is a propylene residue optionally substituted by a hydroxyl group or a C₁-C₆ alkyl radical; R₄, R₅, R₆ and R₇, which are identical or different, represent a hydrogen atom, a C₁-C₆ alkyl or C₁-C₆ hydroxyalkyl radical.

The dyeing composition in accordance with the invention may also contain, in addition to the compound(s) of formula (I) defined above, at least one additional oxidation base which may be chosen from the oxidation bases conventionally used in oxidation dyeing and among which there may be mentioned in particular para-phenylenediamines different from the compounds of formula (I), bisphenylalkylenediamines, para-aminophenols, ortho-aminophenols and heterocyclic bases.

Among the para-phenylene diamines, there may be mentioned more particularly, by way of example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines cited above, there are most particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

Among the bisphenylalkylenediamines, there may be mentioned more particularly, by way of example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-amino-phenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylene-diamine, 1,8-bis(2,5-diaminophenoxy)-3,5-dioxaoctane, and their addition salts with an acid.

Among the para-aminophenols, there may be mentioned more particularly, by way of example, para-aminophenol, 4-amino-3-methylphenol, 4-amino-3-fluoro-phenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols, there may be mentioned more particularly, by way of example, 2-aminophenol, 2-amino-5-methylphenol, 2-amino-6-methylphenol, 5-acetamido-2-aminophenol and their addition salts with an acid.

Among the heterocyclic bases, there may be mentioned more particularly, by way of example, the pyridine derivatives, the pyrimidine derivatives and the pyrazole derivatives.

Among the pyridine derivatives, there may be mentioned more particularly the compounds described for example in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, there may be mentioned more particularly the compounds described for example in German Patent DE 2,359,399 or in Japanese Patents JP 88-169,571 and JP 05 163 124, in European Patent EP 0 770 375 or Patent Application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the pyrazolopyrimidine derivatives such as those mentioned in Patent Application FR-A-2,750,048 and among which there may be mentioned pyrazolo[1,5-a]pyrimidine-3,7-diamine; 2,5-dimethylpyrazolo[1,5-a]pyrimidine-3,7diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-amino-pyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxyethyl)-amino]ethanol, 2-[(7-aminopyrazolo[1,5-a]pyrimidin-3yl)-(2-hydroxyethyl)amino]ethanol, 5,6-dimethyl-pyrazolo[1,5-a]pyrimidine-3,7-diamine, 2,6-dimethyl-pyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine, their tautomeric forms, when a tautomeric equilibrium exists, and their addition salts with an acid.

Among the pyrazole derivatives, there may be mentioned more particularly the compounds described in Patents DE 3,843,892, DE 4,133,957 and Patent Applications WO 94/08969, WO 94/08970, FR-A-2,733,749 and DE-195 43 988 such as 4,5-diamino-1-methylpyrazole, 4,5-diamino-1-(β-hydroxyethyl)pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxy-methylpyrazole, 4,5-diamino-3-hydroxymethyl-1-methyl-pyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their addition salts with an acid.

When they are used, these oxidation bases preferably represent from 0.0005 to 12% by weight approximately of the total weight of the dyeing composition, and still more preferably from 0.005 to 6% by weight approximately of this weight.

The oxidation dyeing compositions in accordance with the invention may also contain one or more couplers and/or one or more direct dyes, in particular for modifying the shades or enriching them with glints.

The couplers which can be used in the oxidation dyeing compositions in accordance with the invention may be chosen from the couplers conventionally used in oxidation dyeing and among which there may be mentioned in particular meta-phenylenediamines, meta-aminophenols, meta-diphenols, naphthols and heterocyclic couplers such as for example indole derivatives, indoline derivatives, pyridine derivatives, indazole derivatives, pyrazolo[1,5-b]-1,2,4-triazole derivatives, pyrazolo[3,2-c]-1,2,4-triazole derivatives, benzimidazole derivatives, benzothiazole derivatives, benzoxazole derivatives, 1,3-benzodioxole derivatives and pyrazolones, and their addition salts with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)-benzene, 2-amino-4-(β-hydroxyethylamino)-1-methoxy-benzene, 1,3-diaminobenzene, 1,3-bis(2,4-diamino-phenoxy)propane, sesamol, α-naphthol, 2-methyl-1-naphthol, 6-hydroxyindole, 4-hydroxyindole, 4-hydroxy-N-methylindole, 6-hydroxyindoline, 6-hydroxybenzomorpholine, 3,5-diamino-2,6-dimethoxypyridine, 1N-β-hydroxyethylamine-3,4-methylenedioxybenzene, 2,6-bis(β-hydroxyethylamino)toluene, 2,6-dihydroxy-4-methylpyridine, 1H-3-methylpyrazol-5-one, 1-phenyl-3-methylpyrazol-5-one, and their addition salts with an acid.

When they are present, the coupler(s) preferably represent from 0.0001 to 10% by weight approximately of the total weight of the dyeing composition and still more preferably from 0.005 to 5% by weight approximately of this weight.

The dyeing composition in accordance with the invention may also contain various adjuvants which are conventionally used in hair-dyeing compositions, such as anionic, cationic, nonionic, amphoteric or zwitterionic surfactants or mixtures thereof, anionic, cationic, nonionic, amphoteric or zwitterionic polymers or mixtures thereof, inorganic or organic thickening agents, antioxidants, penetrating agents, sequestering agents, perfumes, buffers, dispersing agents, conditioning agents such as for example silicones, film-forming agents, preservatives and opacifying agents.

Of course, persons skilled in the art will be careful to choose this or these possible additional compounds such that the advantageous properties intrinsically attached to the oxidation dyeing composition in accordance with the invention are not, or not substantially, impaired by the addition(s) envisaged.

The dyeing composition according to the invention may be provided in various forms, such as in the form of liquids, creams, gels or in any other form appropriate for carrying out a dyeing of keratinous fibers, and in particular human hair.

Another subject of the invention is the use of the compositions of the invention for the oxidation dyeing of keratinous fibers, and in particular human fibers such as hair.

The subject of the invention is also a method of dyeing keratinous fibers, and in particular human keratinous fibers such as hair, using the dyeing composition as defined above.

According to this method, at least one dyeing composition as defined above is applied to the fibers, the color being developed at acidic, neutral or alkaline pH with the aid of an oxidizing agent which is added to the dyeing composition just at the time of use or which is applied separately, simultaneously or sequentially.

According to a preferred embodiment of the dyeing method of the invention, the dyeing composition described above is preferably mixed, at the time of use, with an oxidizing composition containing, in a medium appropriate for dyeing, at least one oxidizing agent present in a sufficient quantity to develop a color. The mixture obtained is then applied to the keratinous fibers and allowed to act for 3 to 50 minutes approximately, preferably 5 to 30 minutes approximately, after which they are washed with. shampoo, rinsed again and dried.

The oxidizing agent may be chosen from oxidizing agents conventionally used for the oxidation dyeing of keratinous fibers, and among which there may be mentioned hydrogen peroxide, urea peroxide, alkali metal bromates, persalts such as perborates and persulfates and enzymes among which there may be mentioned peroxidases, oxidoreductases containing 2 electrons such as uricases and oxygenases containing 4 electrons such as laccases. Hydrogen peroxide is particularly preferred.

The pH of the oxidizing composition containing the oxidizing agent as defined above is such that after mixing with the dyeing composition, the pH of the resulting composition applied to the keratinous fibers preferably varies between 3 and 12 approximately, and still more preferably between 5 and 11. It is adjusted to the desired value by means of acidifying or alkalinizing agents normally used for dyeing keratinous fibers and as defined above.

The oxidizing composition as defined above may also contain various adjuvants conventionally used in hair-dyeing compositions and as defined above.

The composition which is finally applied to the keratinous fibers may be provided in various forms, such as in the form of liquids, creams, gels, or in any other form appropriate for dyeing keratinous fibers, and in particular human hair.

Another subject of the invention is a multi-compartment device or dyeing “kit” or any other multi-compartment packaging system in which a first compartment contains the dyeing composition as defined above and a second compartment contains the oxidizing composition as defined above. These devices may be equipped with a means which makes it possible to deliver the desired mixture onto the hair, by any means known to persons skilled in the art, such as the devices described in Patent FR-2,586,913 in the name of the Applicant.

Finally, the subject of the invention is also the colored product resulting from the oxidation of at least one compound of formula (I) as defined above in the presence of at least one oxidizing agent, and optionally in the presence of at least one coupler and/or of at least one additional oxidation base.

These colored products may also be provided in the form of pigments and may be used as direct dyes for the direct dyeing of hair or may be incorporated into cosmetic products such as for example into make-up products.

The examples which follow are intended to illustrate the invention without, however, limiting the scope thereof.

### EXAMPLES

### Example 1

### 1-(4-Aminophenyl)-4-hydroxypyrrolidine-2-carboxylic acid

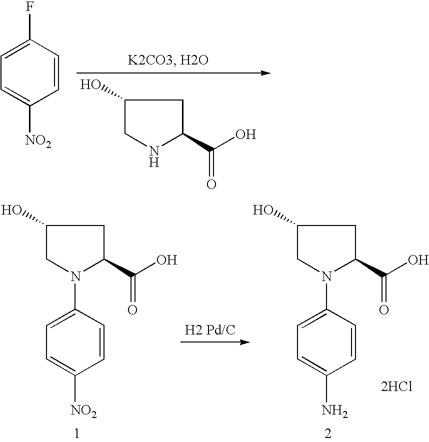

### Synthesis of 1-(4-aminophenyl)-4-hydroxypyrrolidine-2-carboxylic acid (1)

To a solution of 100 ml of water are respectively added 10 g of 4-fluoronitrobenzene (70.9 mmol), 11.75 g of potassium carbonate (85 mmol) and 9.3 g of trans-4-hydroxy-L-proline (70.9 mmol). The reaction medium is then heated under reflux for 16 hours. After cooling to room temperature, the reaction medium is acidified to pH=2 with a 6 N hydrochloric acid solution and then extracted with ethyl acetate (2×100 ml). The organic phases are washed with water (50 ml) and then dried over MgSO4. After filtration and concentration under vacuum, an oil is recovered which is resolubilized in a minimum of ethyl acetate. The desired product is then precipitated by addition of heptane. 16 g (89%) of 1-(4-aminophenyl)-4-hydroxypyrrolidine-2-carboxylic acid 1 are thus obtained in the form of yellow crystals.

¹H NMR (DMSO d6, 200 MHz) 2.26-2.50 (m, 2H), 3.43-3.50 (m, 1H), 3.77-3.85 (m, 1H), 4.55-4.62 (m, 2H), 5.37 (m, 1H), 6.70 (m, 2H), 8.21 (m, 2H)

### Synthesis of compound (2): 1-(4aminophenyl)-4-hydroxypyrrolidine-2-carboxylic acid (2)

2 g of 1-(4-aminophenyl)-4-hydroxypyrrolidine-2-carboxylic acid 1 (7.93 mmol), 1 g of 10% Pd/C (54% moist), 40 ml of ethanol, and 8 ml of cyclohexene are introduced, with stirring, into a 250 ml three-necked round-bottomed flask under a nitrogen atmosphere. The reaction medium is heated under reflux for 5 hours until the 1-(4-aminophenyl)-4-hydroxypyrrolidine-2-carboxylic acid 1 disappears (checked by TLC: NH₄OH: 6/CH₂Cl₂ 47/MeOH 47). The medium is filtered in the hot state in a flask containing 100 ml of diisopropyl ether, 50 ml of isopropanol and 7.2 ml of a hydrochloric ethanol solution (corresponds to 4 equivalents of HCl). A precipitate appears. It is filtered, washed with petroleum ether and then dried under vacuum in the presence of potassium hydroxide. 0.77 9 (33%) of 1-(4-aminophenyl)-4-hydroxypyrrolidine-2-carboxylic acid (2) dihydrochloride is thus obtained in the form of a light beige solid.

¹H NMR (DMSO d6, 200 MHz) 2.50-2.85 (m, 2H), 3.68-3.95 (m, 2H), 4.60-4.64 (m, 1H), 4.75-4.85 (m, 1H), 6.89 (d, 2H), 7.44 (d, 2H)

### Example 2

### Dyeing Composition

The following dyeing compositions in accordance with the invention were prepared:

| Example | Ex. 1 |
|---|---|
| 1-(4-Aminophenyl)-4- | 6 10⁻³ mol |
| hydroxypyrrolidine-2-carboxylic acid | |
| 1-Beta-hydroxyethyloxy-2,4- | 6 10⁻³ mol |
| diaminobenzene, 2HCl | |
| Common dye carrier | (*) |
| Demineralized water qs | 100 g |

(*) Common dye carrier:

| Polyglycerolated oleyl alcohol | 4.0 | g |
|---|---|---|
| containing 2 mol of glycerol | | |
| Polyglycerolated oleyl alcohol | 5.69 | g A.S. |
| containing 4 mol of glycerol, containing | | |
| 78% of active substances (A.S.) | | |
| Oleic acid | 3.0 | g |
| Oleyl amine containing 2 mol of ethylene | 7.0 | g |
| oxide, sold under the trade name | | |
| ETHOMEEN O12 ® by the company AKZO | | |
| Diethylaminopropyl laurylamino | 3.0 | g A.S. |
| succinamate, sodium salt containing 55% | | |
| of A.S. | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9.0 | g |
| Sodium metabisulfite as an aqueous | 0.455 | g A.S. |
| solution containing 35% of A.S. | | |
| Ammonium acetate | 0.8 | g |
| Antioxidant, sequestrant | q.s. | |
| Perfume, preservative | q.s. | |
| Aqueous ammonia containing 20% of NH₃ | 10.0 | g |

At the time of use, each dyeing composition is mixed with an equal quantity of an oxidizing composition consisting of a solution of hydrogen peroxide at 20 volumes (6% by weight) and having a pH of about 3.

Each mixture obtained has a pH of about 9.5 and is applied for 30 minutes to locks of natural gray hair which is 90% white. The locks of hair are then rinsed, washed with a standard shampoo and then dried.

The locks of hair were dyed in the shades presented in the table below:

| EXAMPLE | SHADE ON NATURAL HAIR |
|---|---|
| Ex. 1 | Slightly matt blue |

1. A composition for the oxidation dyeing of keratinous fibers, and in particular human keratinous fibers such as hair, comprising, in a medium appropriate for dyeing,
at least one oxidation base chosen from compounds of the following formula (I), and/or their addition salts with an acid 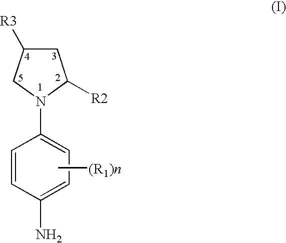
in which:
R₁ represents a halogen atom; a linear or branched C₁-C₇ carbon chain, which is saturated or which may contain one or more double bonds and/or one or more triple bonds, which may be in the form of a 3- to 6-membered ring, it being possible for one or more carbon atoms of the chain to be replaced by an oxygen, nitrogen or sulfur atom, by an SO₂ group or by a halogen atom, the radical R₁ not containing a peroxide bond, or a diazo, nitro or nitroso radical;
R₂ represents a saturated or unsaturated C₁-C₄ carbon chain; a C₁-C₄ alkyl radical substituted with a C₁-C₄ alkoxy radical, an acetoxy radical, an amino radical, a carboxyl radical, a carbamoyl radical, a (C₁-C₄) mono- or dialkylcarbamoyl radical, a (C₁-C₄)alkoxycarbonyl radical, a C₁-C₆ monohydroxyalkoxy radical or with a C₂-C₆ polyhydroxyalkoxy group; a C₁-C₄ monohydroxyalkyl radical; a C₁-C₆ polyhydroxyalkyl radical; a C₁-C₄ aminoalkyl radical in which the amine is mono- or disubstituted with a C₁-C₄ alkyl radical, an acetyl radical, a C₁-C₄ monohydroxyalkyl radical or a C₂-C₆ polyhydroxyalkyl radical; a C₂-C₄ polyaminoalkyl radical; a C₂-C₄ alkyl radical substituted with at least one amino group and at least one hydroxyl group; a carboxyl radical; a carbamoyl radical; a (C₁-C₄) mono- or dialkylcarbamoyl radical; a (C₁-C₄)alkoxycarbonyl radical; a (C₁-C₄) alkylcarbonyl radical,
R₃ represents a hydroxyl radical; an amino radical; a saturated or unsaturated C₁-C₄ carbon chain; a radical —OR₄ in which R₄ represents a saturated or unsaturated C₁-C₄ alkyl radical which may be substituted with one or more radicals chosen from the group consisting of a halogen atom, a hydroxyl, a C₁-C₂ alkoxy, an amino or a C₁-C₂ aminoalkyl radical; a radical —NR₅R₆ in which R₅ and R₆ represent, independently of each other, a hydrogen atom, a C₁-C₄ alkyl radical, a C₁-C₄ alkyl radical substituted with one or more radicals chosen from the group consisting of a halogen atom, a hydroxyl, a C₁-C₂ alkoxy, an amino or a C₁-C₂ aminoalkyl radical.
n is between 0 and 2, it being understood that when n is equal to 2, then the radicals R₁ may be identical or different, with the exception of the compound 1-(3-methyl-4-aminophenyl)-4-hydroxy-2-methylpyrrolidone and the compound 1-(4-aminophenyl)-4-hydroxy-2-hydroxymethylpyrrolidone.

2. The composition as claimed in claim 1, in which the radical R₁ of formula (I) is chosen from a chlorine or bromine atom, a methyl, ethyl, isopropyl, vinyl, allyl, methoxymethyl, hydroxymethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy, or 2-hydroxyethyloxy radical.

3. The composition as claimed in claim 1 or 2, in which the radical R₂ of formula (I) is chosen from the hydroxymethyl radical, aminomethyl radical, carboxyl radical, carbamoyl radical, 2-hydroxyethyloxymethyl radical, 2-hydroxyethylaminomethyl radical, and methoxymethyl.

4. The composition as claimed in one of claims 1 to 3, in which the radical R₃ of formula (I) is chosen from a hydroxyl radical, an acetoxy radical, an amino radical, a methylamino radical, a dimethylamino radical, a hydroxyethylamino radical, a hydroxyethyloxy radical, and an acetamido radical.

5. The composition as claimed in any one of claims 1 to 4, in which n is 0 or 1.

6. The composition as claimed in any one of claims 1 to 5, in which the compound of formula (I) is chosen from the group consisting of:

| Formula | Nomenclature | Formula | Nomenclature |
|---|---|---|---|
| 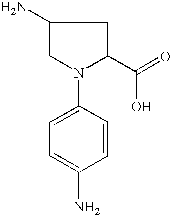 | 4-Amino-1-(4-amino-phenyl)- pyrrolidine-2-carboxylic acid | 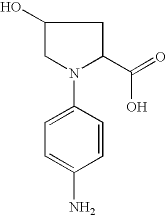 | 1-(4-Amino-phenyl)-4-hydroxy- pyrrolidine-2-carboxylic acid |
| 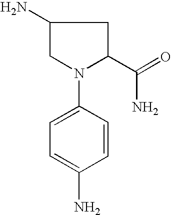 | 4-Amino-1-(4-amino-phenyl)- pyrrolidine-2-carboxylic acid amide | 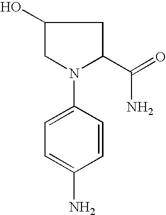 | 1-(4-Amino-phenyl)-4-hydroxy- pyrrolidine-2-carboxylic acid amide |
| 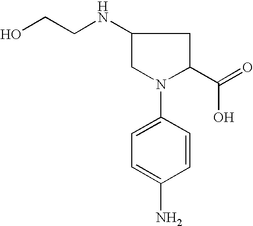 | 1-(4-Amino-phenyl)-4-(2- hydroxy-ethylamino)- pyrrolidine-2-carboxylic acid | 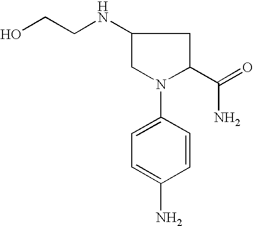 | 1-(4-Amino-phenyl)-4-(2- hydroxy-ethylamino)- pyrrolidine-2-carboxylic acid amide |
| 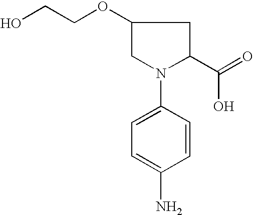 | 1-(4-Amino-phenyl)-4-(2- hydroxy-ethoxy)-pyrrolidine-2- carboxylic acid | 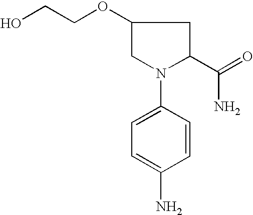 | 1-(4-Amino-phenyl)-4-(2-hydroxy- ethoxy)-pyrrolidine-2-carboxylic acid amide |
| 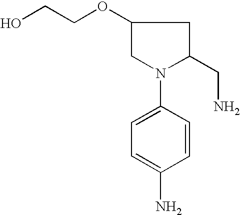 | 2-[5-Aminomethyl-1-(4- amino-phenyl)-pyrrolidin-3- yloxy]-ethanol | 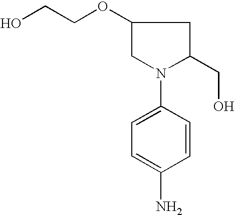 | 2-[1-(4-Amino-phenyl)-5- hydroxymethyl-pyrrolidin-3- yloxy]-ethanol |
| 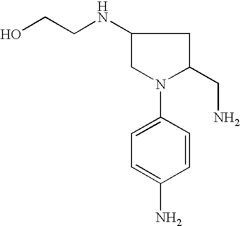 | 2-[5-Aminomethyl-1-(4- amino-phenyl)-pyrrolidin-3- ylamino]-ethanol | 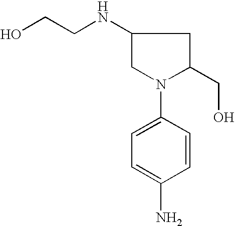 | 2-[1-(4-Amino-phenyl)-5- hydroxymethyl-pyrrolidin-3- ylamino]-ethanol |
| | | 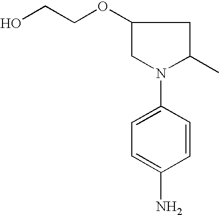 | 2-[1-(4-Amino-phenyl)-5- methyl-pyrrolidin-3-yloxy]- ethanol |
| 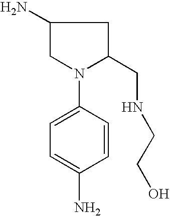 | 2-{[4-Amino-1-(4-amino- phenyl)-pyrrolidin-2- ylmethyl]-amino}-ethanol | 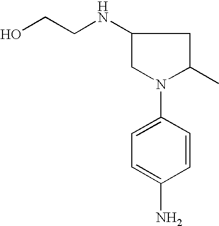 | 2-[1-(4-Amino-phenyl)-5- methyl-pyrroidin-3-ylamino]- ethanol |
| 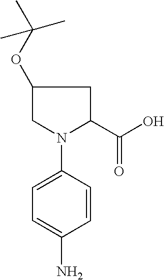 | 1-(4-Amino-phenyl)-4-tert- butoxy-pyrrolidine-2- carboxylic acid | 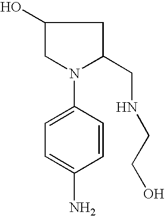 | 1-(4-Amino-phenyl)-5-[(2- hydroxy-ethylamino)-methyl]- pyrrolidin-3-ol |
| 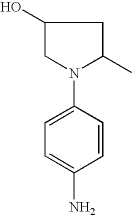 | 1-(4-Amino-phenyl)-5- methyl-pyrrolidin-3-ol | 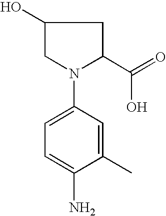 | 1-(4-Amino-3-methyl-phenyl)- 4-hydroxy-pyrrolidine-2- carboxylic acid |
| 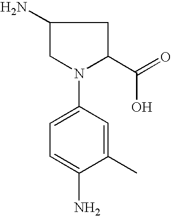 | -Amino-1-(4-amino-3-methyl- phenyl)-pyrrolidine-2- carboxylic acid | 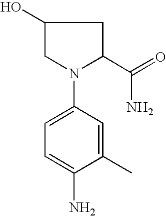 | 1-(4-Amino-3-methyl-phenyl)- 4-hydroxy-pyrrolidine-2- carboxylic acid amide |
| 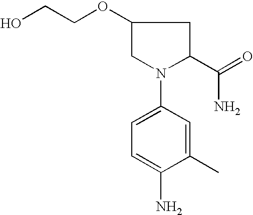 | 1-(4-Amino-3-methyl- phenyl)-4-(2-hydroxy- ethoxy)-pyrrolidine-2- carboxylic acid amide | 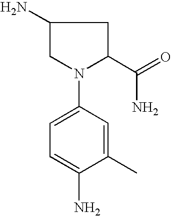 | 4-Amino-1-(4-amino-3-methyl- phenyl)-pyrrolidine-2- carboxylic acid amide |
| 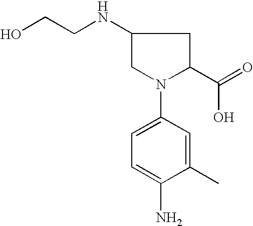 | 1-(4-Amino-3-methyl- phenyl)-4-(2-hydroxy- ethylamino)-pyrrolidine-2- carboxylic acid | 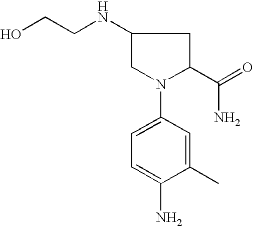 | 1-(4-Amino-3-methyl-phenyl)- 4-(2-hydroxy-ethylamino)- pyrrolidine-2-carboxylic acid amide |
| 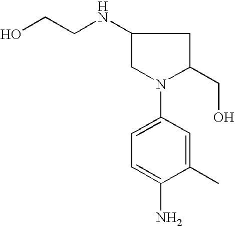 | 2-[1-(4-Amino-3-methyl- phenyl)-5-hydroxymethyl- pyrrolidin-3-ylamino]-ethanol | 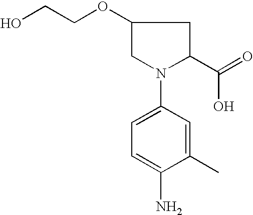 | 1-(4-Amino-3-methyl-phenyl)- 4-(2-hydroxy-ethoxy)- pyrrolidine-2-carboxylic acid |
| 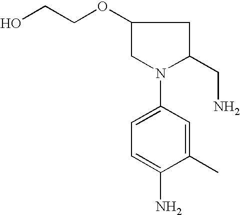 | 2-[5-Aminomethyl-1-(4- amino-3-methyl-phenyl)- pyrrolidin-3-yloxy]-ethanol | 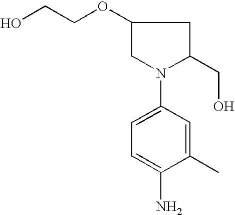 | 2-[1-(4-Amino-3-methyl- phenyl)-5-hydroxymethyl- pyrrolidin-3-yloxy]-ethanol |
| 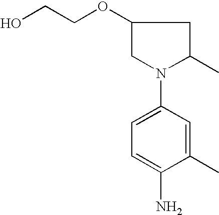 | 2-[1-(4-Amino-3-methyl- phenyl)-5-methyl-pyrrolidin- 3-yloxy]-ethanol | 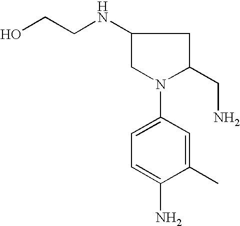 | 2-[5-Aminomethyl-1-(4-amino- 3-methyl-phenyl)-pyrrolidin-3- ylamino]-ethanol |
| 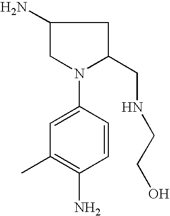 | 2-{[4-Amino-1-(4-amino-3- methyl-phenyl)-pyrrolidin-2- ylmethyl]-amino}-ethanol | 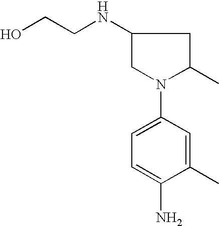 | 2-[1-(4-Amino-3-methyl- phenyl)-5-methyl-pyrrolidin-3- ylamino]-ethanol |
| 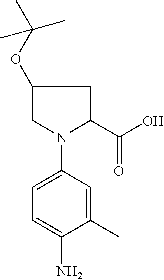 | 1-(4-Amino-3-methyl- phenyl)-4-tert-butoxy- pyrrolidine-2-carboxylic acid | 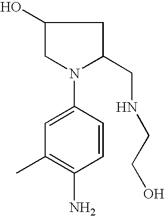 | 1-(4-Amino-3-methyl-phenyl)- 5-[(2-hydroxy-ethylamino)- methyl]-pyrrolidin-3-ol |

and their addition salts with an acid.

7. The composition as claimed in any one of claims 1 to 6, characterized in that the compound(s) of formula (I) represent from 0.0005 to 12% by weight of the total weight of the dyeing composition.

8. The composition as claimed in any one of claims 1 to 7, characterized in that it contains, in addition to the compound(s) of formula (I), at least one additional oxidation base chosen from para-phenylenediamines different from the compounds of formula (I), bis-phenylalkylenediamines, para-aminophenols, ortho-aminophenols and heterocyclic bases.

9. The composition as claimed in any one of claims 1 to 8, characterized in that the additional oxidation base(s) represent from 0.0005 to 12% by weight of the total weight of the dyeing composition.

10. The composition as claimed in any one of claims 1 to 9, characterized in that it contains at least one coupler and/or at least one direct dye.

11. The composition as claimed in claim 10, characterized in that the couplers are chosen from meta-phenylenediamines, meta-aminophenols, meta-diphenols, naphthols and heterocyclic couplers.

12. The composition as claimed in either of claims 10 and 11, characterized in that the coupler(s) represent from 0.0001 to 10% by weight of the total weight of the dyeing composition.

13. The composition as claimed in any one of claims 1 to 12, characterized in that the addition salts with an acid of the compounds of formula (I) are chosen from the hydrochlorides, hydrobromides, sulfates, citrates, succinates, tartrates, lactates and acetates.

14. The use of the composition as claimed in any one of claims 1 to 13 for the oxidation dyeing of keratinous fibers, and in particular human fibers such as hair.

15. A method for the oxidation dyeing of keratinous fibers, and in particular human keratinous fibers such as hair, characterized in that the composition as defined in any one of claims 1 to 13, and an oxidizing agent are applied to the fibers, the oxidizing agent being added to the composition at the time of use or applied to the fibers separately, simultaneously or sequentially.

16. The method as claimed in claim 13, characterized in that the oxidizing agent is chosen from hydrogen peroxide, urea peroxide, alkali metal bromates, persalts, peracids, and oxidoreduction enzymes.

17. A multicompartment device comprising a first compartment containing the composition as defined in any one of claims 1 to 13 and a second compartment containing an oxidizing composition.

18. A colored product which can be obtained by reacting the composition as defined in any one of claims 1 to 13 with an oxidizing agent.
